(19) United States
(12) Patent Application Publication
(10) Pub. No.: US 2023/0190104 A1
(43) Pub. Date: Jun. 22, 2023

(54) AUTOMATED MAPPING AND/OR SIGNAL PROCESSING RESPONSIVE TO CARDIAC SIGNAL FEATURES

(71) Applicant: CARDIOINSIGHT TECHNOLOGIES INC., INDEPENDENCE, OH (US)

(72) Inventors: QINGGUO ZENG, SOLON, OH (US); QING LOU, SOLON, OH (US); RAHSEAN K. ELLIS, ORANGE PARK, FL (US); TIMOTHY G. LASKE, SHOREVIEW, MN (US)

(21) Appl. No.: 18/054,391

(22) Filed: Nov. 10, 2022

Related U.S. Application Data
(60) Provisional application No. 63/292,525, filed on Dec. 22, 2021.

(51) Int. Cl. A61B 5/00; A61B 5/24

(52) U.S. Cl. CPC A61B 5/0036; A61B 5/24

(57) ABSTRACT

A computer-implemented method includes identifying respective heartbeat intervals based on electrophysiological data representative of cardiac electrophysiological signals measured over a time interval. The method includes analyzing the cardiac electrophysiological signals over at least a portion of the time interval. The method also includes generating a map on a surface of interest and/or performing automated signal processing based on the cardiac electrophysiological signals for heartbeat intervals, in which the map is generated and/or the automated signal processing is performed automatically responsive to the analysis of the cardiac electrophysiological signals.

### CROSS-REFERENCE TO RELATED APPLICATIONS

This application claims the benefit of U.S. Provisional Patent Application No. 63/292,525, filed Dec. 22, 2021, which is incorporated herein by reference in its entirety.

### FIELD

The present technology is generally related to automated mapping and/or tagging responsive to cardiac signal features.

### BACKGROUND

Electrophysiology procedures are used to analyze, diagnose and/or treat cardiac electrical activities. Electrophysiology procedures usually take place in an electrophysiology (EP) lab or a catheterization (Cath) lab at a hospital or other medical facility. For example, an EP mapping procedure can be performed in an invasive procedure in which one or more electrode catheters are placed in or on the heart to measure electrophysiology signals. In an additional or alternative example, the EP mapping procedure may be performed using a non-invasive arrangement of electrodes distributed across an outer surface of the patient's body (e.g., on the thorax). In a given EP procedure, the user is tasked with entering respective inputs to control system parameters, acquire and process measured data as well as to determine and control how to generate relevant maps.

### SUMMARY

The techniques of this disclosure generally relate to using analysis of electrophysiological data to automate portions of an electrophysiological mapping and monitoring process.

In one aspect, the present disclosure provides a computer-implemented method that includes computer-implemented method includes identifying respective heartbeat intervals based on electrophysiological data representative of cardiac electrophysiological signals measured over a time interval. The method includes analyzing the cardiac electrophysiological signals over at least a portion of the time interval. The method also includes generating a map on a surface of interest and/or performing automated signal processing based on the cardiac electrophysiological signals for heartbeat intervals, in which the map is generated and/or the automated signal processing is performed automatically responsive to the analysis of the cardiac electrophysiological signals. One or more non-transitory machine-readable medium can be configured to store data and instructions, in which the instructions are programmed to perform the method.

In another aspect, the disclosure provides a system. The system includes a sensing system having one or more electrodes adapted to measure cardiac electrophysiological signals. A computing apparatus includes non-transitory memory to store data and instructions executable by a processor thereof. The stored data can include electrophysiological data representative of the measured cardiac electrophysiological signals measured over one or more time intervals and geometry data representing anatomy of the patient spatially, and locations of the electrodes in three-dimensional space. The instructions can be programmed to perform the method described above.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a block diagram of an example system that can be implemented for measuring and monitoring electrophysiological signals.

FIG. 2 is a block diagram of an example electrophysiological data analyzer that can be used in the system of FIG. 1.

FIG. 3 is a block diagram of an example feature calculator that can be used in the system of FIG. 1.

FIG. 4 is a flow diagram of an example method that can be implemented for measuring and monitoring electrophysiological signals.

### DETAILED DESCRIPTION

This disclosure relates to a mapping and monitoring system configured to acquire signals and generate maps, such as can be implemented in electrophysiology (EP) studies and procedures. For example, the mapping and monitoring system can determine signal features associated with cardiac electrophysiological signals over a time interval based on one or more parameters. The system can generate a map on a surface of interest and/or perform automated signal processing based on the cardiac electrophysiological signals acquired for one or more intervals that include the computed signal features. The map can be generated or the other automated signal processing can be performed automatically responsive to the signal features, and which signal features can include changes to electrical activity of cardiac tissue and rhythm changes.

As an example, during EP studies and procedures, manual configurations and settings are often utilized throughout the EP procedure. In addition to adding complexity to the overall study, such manual processes may be insufficient in providing real-time feedback during the procedure. Accordingly, the mapping and navigation system of the present disclosure is configured to automatically detect electrophysiological signals and signal features based on measured EP signals. As described herein, the measured EP signals can be obtained invasively, non-invasively, or both invasively, non-invasively. In an example, the mapping and navigation system includes an integrated single-beat detector configured to identify respective heartbeats in the measured signals (e.g., based on invasive and/or non-invasive measurement methods). The system also is configured to implement automated data and map analysis, which enables feedback with beat-to-beat resolution. Moreover, methods such as machine learning or template matching can be performed on measured and/or reconstructed EP signals to automatically detect cardiac rhythms and cycle length as well as changes in rhythm and cycle length that can occur over respective intervals. In some examples, electrocardiographic maps can be generated automatically responsive to the detected changes in cardiac rhythms, cycle length and/or other signal features. During time intervals experiencing stable rhythms, signal processing functions (e.g., signal averaging) can be performed to further improve signal quality of the EP signal measurements. An output (e.g., a report) can also be generated for an EP study performed that summarizes procedure steps in the EP study. For example, the report can summarize features in EP signals measured during the study as well as information derived from the measured signals. The report further can store actions and commands entered during the study by the user through one or more user input device. By automating these and other part of the mapping and monitoring process, users can focus more on EP interpretation and patient care. As a result, the amount of time for the EP process can be reduced and the overall user experience improved.

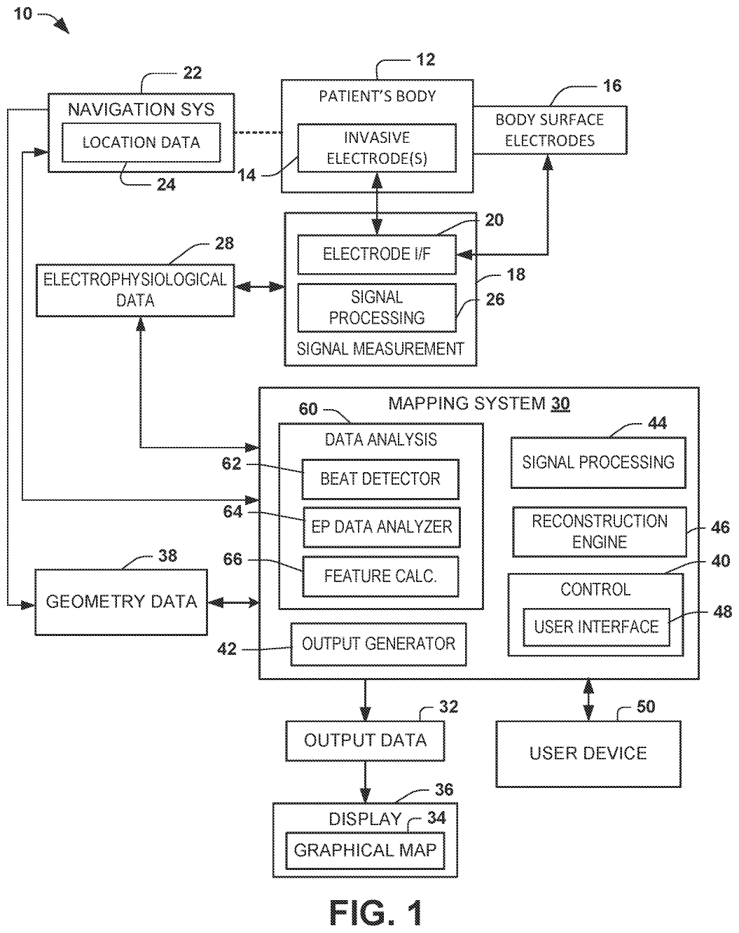

FIG. 1 depicts an example of a system 10 for monitoring and mapping electrophysiological measurements from a patient 12. The system 10 includes one or more invasive electrodes 14. The system 10 can additionally, or alternatively, include body surface electrodes 16. For purposes of consistency, the following description describes the system as having both invasive and body surface electrodes 14 and 16. However, in other examples, the system 10 may include only invasive electrodes 14 or only body surface electrodes 16. The respective electrodes 14, 16 are coupled to a signal measurement device 18.

As an example, each of the electrodes 14, 16 is coupled to the signal measurement device 18 through a respective electrically conductive channel (e.g., including electrically insulated wires and/or traces) to communicate electrophysiological signals measured from the patient's body. The electrically conductive channels for respective electrodes 14 and 16 can also include an arrangement of connectors configured to couple to respective connectors (e.g., male and female connectors) of an electrode interface 20 of the measurement device 18. In other examples, the electrodes 14, 16 may be coupled to the electrode interface 20 through other forms of communication (e.g., optical fiber or wireless leads). The electrode interface 20 can measure unipolar, bipolar or a combination of unipolar and bipolar electrophysiological signals depending on the configuration of the measurement device 18 and processing of the signals measured by the electrodes 14 and 16.

As an example, the one or more invasive electrodes 14 can be coupled to or otherwise carried by an invasive device, such as an electrophysiology probe. The probe can be a catheter that is moveable within the patient's body 12, such that the position of the probe and associated electrode(s) 14 can vary. For example, a cardiac catheter can be inserted into a femoral vein (or other known entry point) and advanced to a position within the patient's heart. Alternatively, the probe and electrode(s) 14 can be configured to measure electrophysiological signals on an outer surface of the patient's heart. Thus, the signals measured by the invasive electrodes 14 depend on where the probe is positioned within the patient's body 12. The probe (or other invasive device) may be moved manually, robotically assisted or fully robotically.

In some examples, the system 10 also includes a navigation system 22 configured to localize the spatial position of the invasive device and electrode 14. The spatial position of the electrode 14 (or associated invasive device) can be stored in memory as location data 24. The location data 24 thus represents a three-dimensional spatial position (e.g., spatial coordinates) of the electrode 14. Alternatively, the location data 24 can represent the location of a sensor or other known location on the probe carrying the electrode(s), and the spatial location of each electrode 14 can be derived readily from the location data 24. The spatial location of the electrode(s) 14 can be with respect to the patient's body or a coordinate system of the navigation system 22. As described below, for example, the spatial location of the invasive electrode 14, which is described by or derived from the location data 24, can be registered with respect to anatomical geometry of the patient's body 12. The registration can be repeated in response to detecting changes in the location data as the electrode is moved within the patient's body. In some examples, the navigation system 22 can also generate the location data 24 to include the location of one or more of the non-invasive electrodes 16, which are distributed across an outer surface of the patient's body (e.g., on the thorax).

Useful examples of the navigation system 22 include the STEALTH STATION navigation system (commercially available from Medtronic), the CARTO XP EP navigation system (commercially available from Biosense-Webster) and the ENSITE NAVX visualization and navigation technology (commercially available from St. Jude Medical); although other navigations systems could be used to provide the navigation data representative of the spatial position for the invasive electrode 14 and associated probe. Another example of a navigation system that can be utilized to localize the position of the invasive electrodes is disclosed in U.S. Pat. No. 10,323,922, issued Jun. 18, 2019 Aug. 29, 2014, and entitled LOCALIZATION AND TRACKING OF AN OBJECT, which is incorporated herein by reference. For example, a probe (e.g., catheter) can include one or more electrodes 14 disposed at known locations with respect to the probe. The probe can be used to position each such electrode 14 with respect to the heart and the navigation system 22 can determine corresponding three-dimensional coordinates for the electrode(s) 14 that is represented by the location data 24.

The number and placement of invasive electrodes 14 can vary depending upon the type of catheter or other invasive device to which the electrodes are coupled. In a further example, the invasive electrode(s) can be contact electrodes that measure signals from a surface of an object that the electrode physically engages or contacts. Alternatively, the invasive electrode(s) 14 can be non-contact electrodes that measure signals from a surface of an object while the electrode is spatially apart from (e.g., no physical contact between the electrode and the surface being measured). Such non-contact electrodes can perform mapping from the body surface or from within a cardiac chamber.

The body surface electrodes 16 include a distributed arrangement of multiple electrodes (e.g., about 50, 100, 250 or more sensors) positioned on an outer surface of the patient's body 12. In an example, the body surface electrodes 16 are distributed completely around the thorax, such as can be mounted to a wearable garment (e.g., vest) in which each of the electrodes has a known location in a given coordinate system. For example, body surface electrodes 16 can be implemented as a non-invasive type of sensor apparatus as disclosed in U.S. Patent Publication No. 2013/0281814, entitled Multi-Layered Sensor Apparatus. Other configurations and numbers of body surface electrodes 16 could be utilized in other examples.

As described above, the electrode interface 20 has respective inputs coupled to each of the electrodes 14 and 16. The signal measurement device 18 can also include signal processing circuitry and/or software function 26 configured to process electrical signals measured by the respective electrodes 14, 16. The signal processing circuitry 26 can be implemented as hardware and/or software, such as including a digital signal processor and other processing circuitry and machine readable instructions (executable by a processor) configured to remove noise (e.g., line noise) and convert the received signals into a desired format for storing the measured electrophysiological signals as electrophysiological data 28. The signal processing circuitry 26 can also add channel information (e.g., to specify electrode number or location), add timestamps (e.g., to specify the time or each measurement sample) or perform other signal processing functions that may be desired. The electrophysiological data 28 thus can include signal measurement values for each sample, including signal morphology, as well as additional information, such as time stamps and channel information. In an example, the signal processing circuitry 26 can extract signal morphology features, such as cycle length, dominant frequency, waveform geometry the like, and store such extracted signal features with the electrophysiological data 28.

The system 10 includes one or more processors configured to access memory that stores data. The processor(s) can access and execute instructions corresponding to the functions and methods implemented by the mapping system 30. The mapping system 30 thus includes instructions executable by the one or more processors of the computer apparatus to perform the functions described herein. In the example of FIG. 1, the mapping system includes an output generator 42, signal processing function 44 and data analysis function 60. The mapping system 30 also includes a control function 40 configured to control one or more processing, analysis and mapping functions implemented by the system 30.

The output generator 42 is programmed to generate output data 32 that can be rendered as a graphical map 34 on the display 36 to graphically visualize EP signals on a surface of interest. For example, the output generator 42 is programmed to generate an EP map based on the reconstructed signals (generated by reconstruction engine 46) and geometry data 38. By including reconstructed electrical signals (e.g., derived from both non-invasive measurements and invasively measured signals) in a respective map, the respective map can more accurately represent cardiac electrophysiological signals on the surface of interest.

As disclosed herein, the surface of interest may be an epicardial surface, an endocardial surface, a combination of an epicardial or endocardial surfaces or a full three dimensional rendering of the heart tissue, including epicardial, endocardial and transmural myocardium throughout the heart. Additionally, or alternatively, the surface of interest can be a cardiac envelope, such as a virtual surface residing between the center of a patient's heart and the body surface where the electrodes are positioned. The surface of interest may encompass the entire cardiac surface or one or more regions (epicardial or endocardial) of interest. The output generator 42 thus is configured to provide the output data 32 to the display 36 to visualize one or more electrocardiographic maps 34 as well as other electrical information derived from the EP data 28 and geometry data 38. The output generator 42 can also provide information in other display formats to provide guidance to the user representative of and/or derived from electrical activity that may be measured by any combination of the electrodes 14 and 16. For example, the mapping system 30 may further use the output generator 42 to provide guidance to help a user move the invasive electrode 14 (or other interventional device) to a location of interest (e.g., on or near a region of interest of the heart) based on the electrophysiological data 28 and the geometry data 38. As a further example, the control function 40 can include instructions to control the output generator to instruct the user to move a catheter or other invasive device (e.g., by generating a notification to reposition the catheter or probe) based upon detected changes in rhythms or other signal features that are automatically detected. This can help improve the fidelity of mapping and/or improve delivery of a desired intervention (e.g., therapy, ablation, CRT, application of a bioactive agent or other intervention) at an effective site.

The geometry data 38 includes electrode geometry data and anatomical geometry data. The electrode geometry data represents spatial locations of respective body surface electrodes 16 and invasive electrode 14 in three-dimensional space. The anatomical geometry data represents spatial geometry of the surface of interest of the patient in three-dimensional space. The mapping system 30 can further programmed to co-register the electrode geometry data and the anatomical geometry data in a common coordinate system to provide the geometry data. The spatial registration function can implement one or more transforms to align spatially respective data sets for location of the invasive electrodes 14, the location of the body surface electrodes 16 as well as the anatomical geometry for the surface of interest.

As an example, the navigation system 22 generates location data 24 to represent the spatial location of the invasive electrodes 14 in a given coordinate system (e.g., of the navigation system), which may be different from the coordinate system in which the anatomical geometry data is generated. The anatomical geometry data can be derived from imaging data acquired by a three-dimensional medical imaging modality. The medical imaging data can be generated for the patient's body using a medical imaging modality, such as single or multi-plane x-ray, computed tomography (CT), magnetic resonance imaging (MRI), ultrasound, positron emission tomography (PET), single-photon emission computed tomography (SPECT) and the like. The electrode locations and locations of the surface (or surfaces) of interest can be identified in a respective coordinate system of the acquired images through appropriate image processing, including extraction and segmentation. For instance, segmented image data can be converted into a two-dimensional or three-dimensional graphical representation that includes the volume of interest for the patient. Appropriate anatomical or other landmarks, including locations the electrode 14, can be identified in the geometry data 38 to facilitate spatial registration of the electrophysiological data 28. The identification of such landmarks can be done manually (e.g., by a person via image editing software) or automatically (e.g., via image processing techniques). In one example, an anatomical model can be constructed based on imaging data obtained (e.g., by a medical imaging modality) for the patient to provide spatial coordinates for points across the surface of interest. In some cases, in which the electrodes 16 are positioned on the patient's body when the medical image is acquired, spatial coordinates can be provided for the locations where the body surface electrodes 16 are positioned on the outer surface of the patient's body.

In another example, the location of the body surface electrodes 16 can be acquired by a digitizer, manual measurements or another non-imaging based technique, such as including being obtained by the navigation system 22 and included as part of the location data 24. The spatial registration function can provide the geometry data 38 to include the location information for the electrodes 14 and 16 as well as the anatomical geometry all spatially aligned in the common coordinate system. Because the location of the invasive electrode 14 can be moved within the patient's body 12, the corresponding location data 24 can be updated (e.g., in real-time or near-real time) to reflect the current spatial location where the invasive electrical measurement is obtained. Thus, the navigation system 22 can further be programmed to update the geometry data 38 in response to detecting change in the location data 24 for one or more electrodes 14. The location data 24 can also include a time stamp so that the mapping system 30 can programmatically link (e.g., synchronize) a given time instance of the geometry data 38, which includes location of the electrode 14, with respective samples of the electrophysiological signals that are measured.

In the example of FIG. 1, the mapping system 30 also includes a reconstruction engine 46 (e.g., instructions) programmed to compute reconstructed electrophysiological signals for locations on the surface of interest within the patient's body 12. In one example, the reconstruction engine 46 computes the reconstructed signals (e.g., as electrical potentials) on the surface of interest by executing machine-readable instructions (e.g., an algorithm) programmed to reconstruct electrical signals spatially and temporally on to the surface of interest based on the electrophysiological data 28 and the geometry data 38. As described herein, the geometry data 38 includes three-dimensional spatial information representing the surface (or surfaces) of interest describing a surface on to which reconstructed signals are computed (by engine 46) co-registered with respective locations where electrophysiological measurements are made (e.g., by the electrodes 14 and 16). The reconstruction engine 46 can calculate the reconstructed electrical signals on the surface of interest for one or more surfaces of interest over one or more time intervals. The time interval(s) may be selected through a user interface 48 in response to a user input entered by a user device 50 either locally or from a remote location (e.g., mouse, keyboard, touchscreen interface, gesture interface or the like). Alternatively, the time interval(s) can be selected automatically by the data analysis function 60.

As a further example, the reconstruction engine 46 includes code programmed to implement the method of fundamental solutions (MFS). The reconstruction engine 46 thus employs the MFS to solve an inverse problem for computing reconstructed electrical signals on the surface of interest based on the EP data 28 and the geometry data. MFS includes a mathematical representation that spatially relates an influence of the electrophysiological signals measured on the outer surface of the patient's body and the electrophysiological signals measured within the patient's body to the electrophysiological signals on the surface of interest. In an example, the MFS method can implement MFS ECGI similar to that disclosed in U.S. Pat. No. 7,983,743, which is incorporated herein by reference, and further modified to utilize the electrophysiological data that includes measurements from both the invasive and non-invasive electrodes 14 and 16. Other useful examples of inverse algorithms that can be implemented by the reconstruction engine 46 to reconstruct include the boundary element method (BEM), such as disclosed in U.S. Pat. Nos. 6,772,004, and 9,980,660, each of which is incorporated herein by reference. The reconstruction engine 46 further may employ a regularization technique (e.g., Tikhonov regularization) to estimate values for the reconstructed electrical signals on the surface of interest.

In a further example, the control function 40 is configured to control the output generator 42 and/or signal processing function 44 based on the data analysis function 60. In the example of FIG. 1, the data analysis function 60 includes a beat detector function 62, an EP data analyzer function 64 and a feature calculator function 66.

The beat detector function 62 is programmed to analyze the EP data 28 and define a plurality of heartbeat intervals for one or more respective EP signals. The beat detector 62 can detect heartbeats in one signal type or combination of such different signals that can be measured or generated by the system 10. In one example, the EP signals can correspond to a signal measured by one or more of the body surface electrodes 16. In another example, the EP signal(s) used by the beat detector 62 can include signals measured by the invasive electrode 14. In yet another example, the EP signals used by the beat detector 62 can be reconstructed EP signals (e.g., generated by reconstruction engine 46). Various existing heartbeat detection algorithms could be implemented by the beat detector 62, for example the Pan-Tomkins, or modified versions on the intracardiac EP signals. The beat detector 62 can append start and/or stop time information to the EP data 28 for each detected heartbeat, such as by tagging respective beats (e.g., with metadata) in such data or otherwise storing information in memory to specify the respective heartbeats. The beat detector 62 can identify the heartbeat interval automatically, such as described above, or in response to a user input selecting one or more respective intervals of signals (e.g., on a graphical user interface).

The EP data analyzer 64 is programmed to analyze a portion of the EP data 28 defined by respective heartbeat intervals (e.g., as defined by beat detector 62) to determine one or more parameters associated with the respective beats of EP signals over one or more time intervals. The time intervals can be selected in response to user input or correspond to various time intervals for which the EP data 28 has been acquired and encompass the detected heartbeats. The EP data analyzer 64 thus generates the parameters to represent a number of EP signals representative of cardiac electrical activity across the surface of interest. For example the surface of interest can be a cardiac surface of interest such as an endocardial surface, epicardial surface or surface. Alternatively, the surface of interest can be a virtual surface within a patient's body. As mentioned, reconstruction engine can reconstruct the measured EP signals onto the surface of interest. Thus in some examples, the data analyzer 64 can be applied to analyze a particular region (or subregion) of the surface of interest of the patient's heart. Alternatively or additionally, the analysis can be implemented with respect to the entire surface of interest (e.g., the entire cardiac surface) for each of the respective heartbeat intervals.

For example, the EP data analyzer 64 can derive a number of signal parameters for the EP signals, which can include parameters for each heartbeat or parameters that describe signal characteristics over more than one heartbeat. The signal parameters can be low level parameters that describe attributes of respective signal waveforms and may be extracted or derived directly from a given signal waveform. Examples of signal parameters include amplitude, slew rate, frequency components as well as morphological characteristics of one or more components of each heartbeat interval. Alternatively or additionally, the signal parameters can be derived from components of the body-surface or unipolar EP waveform, such as to describe one or more attributes of the P, Q, R, S, T waveform components, such as width of respective segments or intervals, amplitude, slope, number of peaks or parameters that describe a combination of two or more waveform components (e.g., QRS duration and/or morphology, R-R interval, P-P interval, QT duration and/or morphology, etc.). In a further example, each of the different waveform components can be parameterized by a number of signal and/or morphological parameters that can be stored in memory associated with the respective heartbeat interval and signal for which the interval is associated. Such parameters may be stored as part of the EP data 28 (e.g., as signal parameter metadata) or otherwise associated (or linked) with the EP data.

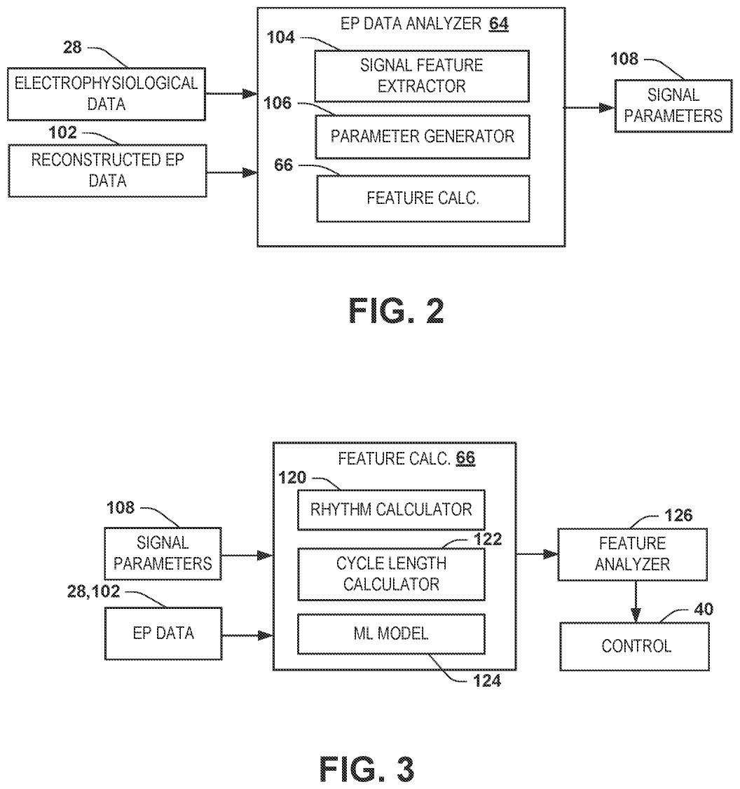

In an example, the EP data analyzer 64 can include a feature calculator 66 programmed to compute one or more signal features associated with the cardiac electrophysiological signals over at least a portion of a time interval, such as based on one or more parameters (e.g., determined by the EP data analyzer 64). The feature calculator 66 can be part of the EP data analyzer 64 (see, e.g., FIG. 2) or it can be implemented as separate program code as shown in FIG. 1. The feature calculator 66 can compute respective features of the EP signals distributed across a region of interest. The region of interest may include a subregion of a cardiac surface up to an entire cardiac surface for which the signals have been measured or reconstructed. In one example, the computed signal features can include cardiac rhythm of the respective signals. The feature calculator 66 further can be configured to evaluate the detected rhythm over multiple heartbeats (e.g., as defined by beat detector 62) to ascertain whether the computed signal features described a normal sinus rhythm or an arrhythmogenic condition. Additionally or alternatively, the feature calculator 66 can be programmed to compute a cycle length and respective frequency components for the respective heartbeat intervals that have been identified. The respective computed features can be stored as part of the EP data 28 (e.g., as feature metadata) or otherwise associated (linked) with the EP data 28. Additionally, feature calculator 66 can be programmed to derive one or more conduction patterns from local bipolar or unipolar electrograms as respective features. As an example, the sequence of activation from the distal to the proximal electrode in a multipolar catheter can be used to describe a beat and determine a change with respect to an existing beat/pattern (e.g., a variation from a baseline beat/pattern).

The control function 40 can be programmed to control the signal processing function 44 and/or the output generator 42 based on analysis of the computed features and/or parameters. For example, the data analysis function 60 can analyze the computed signal features over multiple heartbeat intervals, such as by computing statistics (e.g., mean or variance) associated with such features. In response, the control function 40 can trigger the output generator 42 to generate a corresponding electrocardiographic map on a surface of interest or multiple surfaces based on the cardiac EP data 28 for respective heartbeat intervals that include only the computed signal features. Additionally or alternatively, the control function 40 can trigger the output generator 42 to generate a corresponding electrocardiographic map on a surface of interest or multiple surfaces based on the cardiac EP data 28 for a continuous time interval that includes or encompasses the set of computed signal features. The generation of the maps and/or results of such signal processing can be displayed in the foreground and displayed to the user responsive to the control function 40. Alternatively, the control function can cause the map generation and/or signal processing to be implemented by respective functions running as background processes, and can be selected in response to a user input (e.g., selecting a radio button or other graphical user interface element) and rendered on the display.

As another example, the control function 40 can trigger the output generator 42 to generate a corresponding electrocardiographic map on one or more surfaces of interest in response to detecting changes in one or more signal parameters and/or signal features. In one example, the changes can be detected based on determining a variation in such parameters and/or features with respect to a baseline. The baseline can be generated in response to a user input specifying a normal or baseline signal waveform from which the baseline parameter(s) and/or feature(s) are derived. Alternatively, the baseline can be identified automatically based on analysis of measured EP signals over an extended time period for a given patient relative to known signals for the patient and/or a patient population. Still further the baseline can be determined based on measurements obtained at the start of or during an early phase of an intervention (e.g., prior to ablating tissue). The baseline can represent one or more signal parameters and/or features determined for a normal cardiac rhythm or a cardiac arrhythmia (e.g., a baseline AT or baseline PVC). The control function 40 thus can trigger the output generator 42 to generate a respective map and/or signal processing 26, 44 in response to detecting changes in one or more such baseline parameters and/or features.

In another example, the changes in one or more signal parameters and/or signal features can be detected based on comparing one or more such parameters and/or features relative to one or more respective thresholds. The threshold value can be determined as a percentage change (e.g., a relative threshold) from a prior value of such parameter. Alternatively, the threshold can be a threshold value specifying an absolute value of a feature, parameter or a value representative of a change in such feature or parameter. Each such threshold can be implemented as a default value or be set in response to a user input. Thus, the threshold can be set for a patient cohort or be patient specific. In some examples, to trigger the output generator 42 to automatically generate a map or perform automated signal processing, as described herein, the change can include a combination of more than one change, such as can be defined as by combinatorial logic stored in memory and executed by the control function 40.

As a further example, the signal processing 44 can include an application of one or more signal processing functions 26, 44 to the EP data 28 in response to the analysis of the computed signal features and/or parameters. In an example, the control function 40 can apply signal processing 44 to process the EP data 28 that is stored in memory. In another example, the control function 40 can configure the signal processing 26 of the signal measurement device 18 to adjust the signal processing that is applied to the signals measured from the respective electrodes 14 and/or 16. The signal processing function 26, 44 can include one or more of identification of bad channels, filtering (e.g., notch filter, band pass filter, low pass filter or the like).

Additionally, as described herein, the control function 40 can trigger the reconstruction engine 46 and/or the output generator 42 to generate respective graphical maps of reconstructed EP signals for the surface of interest or a portion thereof responsive to the data analysis 60 of the computed signal features and/or parameters. For example, the output generator 42 can be programmed to generate a graphical map that spatially includes one or more regions of interest up to and including the entire surface (e.g., an entire cardiac surface) for one or more time intervals.

In one example, the time interval for which the map is generated includes one or more heartbeat intervals for which the data analysis function 60 has detected a change in the computed signal features. For example, in response to detecting changes in the cardiac rhythm, the control function 40 can trigger the output generator 42 to generate one or more graphical maps to be provided to the display 36. In some examples, a dialog box can pop up to allow the user to accept or reject the display of proposed graphical map that has been generated. Additionally, the dialog box can include further information about the map, such as including a description of the type of map and the detected signal features that have triggered the map to be generated (e.g., based on the feature metadata). In this way a user can be provided with actionable information more quickly and with fewer manual user input steps. The system may also display a comparison of the two rhythms, for example through subtraction of common elements, in order to identify regions with modified or differing activity.

As a further example, the control function 40 includes instructions programmed to compare the generated map with respect to a reference map and to identify regions of interest and/or differences between the automatically generated map and the reference map based on the comparison. The reference map can be generated for the surface of interest (e.g., the same region as the map being compared) based on cardiac electrophysiological signals for one or more intervals, such as from measurements acquired prior to those used to trigger automatic map generation (e.g., from baseline patient data).

In another example, the data analysis 60 can determine changes in cycle length signal features. In response to detecting such changes in cycle length (e.g., compared to a local or global cycle length threshold), the control function 40 can activate the output generator 42 to provide one or more graphical maps for a region of interest or up to the entire surface of interest responsive to the detected cycle length changes. For example, the detected cycle length changes can be indicative of an arrhythmogenic condition (e.g., atrial fibrillation or other arrhythmia) or a change from an arrhythmia to a normal condition or from a normal cycle to arrhythmia, and the computed signal features can be used to automatically generate a map containing information relevant to the detected condition. As mentioned, a pop-up dialog box can be generated to alert the user that a corresponding map has been generated in response to such cycle length changes, which can be accepted or rejected by the user in response to a user input through the user interface 48. The dialog can also provide information describing the type of signal changes as well as specify a location on a cardiac surface where such changes occurred (e.g., based on feature metadata).

In some examples, in response to the control function 40 detecting stable cardiac activity over a time interval (e.g., for at least a predetermined duration), such as a stable rhythm or stable cycle length over a period of multiple heartbeats determined based on computed signal features (e.g., by feature calculator 66), the control function 40 can invoke the signal processing function 26, 44 to automatically perform certain signal processing functions. For example, the control function 40 can invoke the signal processing function 26 and/or 44 to improve signal quality, such as by implementing signal averaging across a plurality of heartbeat intervals for each of the respective signals. In response to the condition changing from a stable cardiac signal to an unstable condition, the signal averaging (or other signal quality improving function) can be terminated and an additional action taken, such as described above.

In another example, the feature calculator 66 is programmed to apply a trained machine learning (ML) model to the one or more parameters determined (e.g., by EP data analyzer 64) to determine respective signal features of the EP signals for a portion of a time interval. For example, the ML model (e.g., machine-readable instructions executable by a processor) can be pre-trained to automatically detect and classify one or more types signal features, such as a normal rhythm or arrhythmias, such as tachycardia, bradycardia, atrial fibrillation or others. Signal waveforms classified as normal further can be used to determine baseline signal features and/or parameters, which can be stored in memory for the given patient and used to determine variations with respect to the baseline, as described herein.

In some examples, the system 10 can be used during an interventional procedure, such as including delivery of energy (e.g., ablation or electrical stimulation, such as pacing or cardiac resynchronization therapy (CRT)) and/or application of a bioactive agent (e.g., chemical or pharmaceutical and the like). The delivery of energy and/or application of a bioactive agent can be adapted to effect a change tissue function, which change in tissue function can be temporary or permanent according to the objective of the intervention. For example, when a physician is performing ablation, the mapping system 30 can provide feedback to help drive respective ablations (or other interventions). Traditionally, in labs that perform EP, feedback for ablation is obtained using 12-lead ECG and intracardiac catheters; however such traditional approaches are often insufficient to detect meaningful changes in real-time for complex arrhythmias, such as persistent atrial fibrillation. The mapping system 30 can provide enhanced real-time feedback for procedures, such as ablation, CRT or the like, to increase efficacy and improve outcomes.

For example, the mapping system 30 can be configured to provide communication among pacing, ablation and/or other interventions and real-time mapping (e.g., electrocardiographic imaging (ECGI)). The non-invasive component of the real-time mapping can further be programmed to detect and determine when a given ablation occurs and automatically analyze cardiac signals and maps derived from such signal before and after the given ablation. Additionally, the data analysis 60 can be programmed to determine whether one or more signal features (e.g., computed by feature calculator 66) for a region of interest of the heart have changed in response to the given intervention and, as disclosed herein, automatically generate an ECGI map to display information about the change, which can be rendered as a graphical map 34 on display 36.

For example, the EP data analyzer 64 is programmed to extract parameters extracted from the EP signals (e.g., measured or reconstructed EP signal). The feature calculator 66 can compute features based on such signal parameters. In an example, the feature calculator can compute such features from the parameters by applying an ML model trained to compute one or more features, including signal morphology, cycle length, dominant frequency, earliest activation region, slowest conduction region, and conduction patterns (e.g., focal and reentrant) as well as changes in such features. If the data analysis detects changes in the electrophysiological condition for one or more regions of interest responsive to the intervention (e.g., ablation, stimulation, application of bioactive agent, etc.), the data analysis 64 can tag the respective region(s) to indicate that the region under intervention is part of a critical circuit path or source sustaining the arrhythmia, and thus may warrant additional ablation or other intervention in the region. If no such changes are observed responsive to the ablation, the mapping and navigation system can suggest another region of interest or indicate the need to ablate at the same region with an increased power or duration.

In some examples, the output generator 42 can also be programmed to generate a report that describes steps performed in the EP procedure by the user. The report can include data described in each of the steps implemented by the user in response to the respective user input as well as data describing the computed signal feature/characteristics and/or changes thereof associated with the cardiac EP signals over relevant time intervals. The acceptance or rejection of automated maps or signal processing functions applied by the control function 40 can also be stored in memory and included as part of the report to document information presented to the user during the procedure. The reports can be stored in memory and/or sent to one or more other users (e.g., by email, text messaging or other messaging protocol) for further review/analysis and/or archiving thereof.

FIG. 2 depicts an example of the EP data analyzer 64. The EP data analyzer 64 is configured to generate signal parameters 108 based on EP data 28 which can include measured EP data, reconstructed EP data or a combination of measured or reconstructed EP data. The EP data analyzer includes a signal feature extractor 104 and a parameter generator 106. The signal feature extractor 104 is configured to analyze one or more respective EP signals and identify corresponding signal characteristics within one or more heartbeat intervals of the respective signals. For example, signal features can include changes in local or global cardiac cycle length, changes in activation patterns, changes in signal morphology, changes in frequency components and/or any other modifications to the electrical characteristics of the region of interest. An example of such a change could be the termination of atrial fibrillation to an atrial tachycardia, having both a slower cycle length and one that is consistent across the chamber/region of interest. The parameter generator 106 is configured to process the extracted features and to generate the signal parameters 108. Thus, as described herein, the signal parameters characterize different attributes of the respective signals, such as may include a value representative of respective signal features including amplitude (e.g., a normalized amplitude), a frequency or period, as well as corresponding parameters for respective waveform components of the respective heartbeat intervals. The features of such waveform components (e.g., the Q, R, S, T and/or P waveform components) may include morphological as well as other respective segments and/or intervals of the waveform components within a respective heartbeat or between sequential beats. The resulting signal parameters 108 can be stored in memory for further processing by the feature calculator function 66, as described herein.

FIG. 3 depicts an example of the feature calculator function 66, which receives as respective inputs the signal parameters 108 as well as the EP data 28 and/or 102. The feature calculator 66 includes a rhythm calculator 120, a cycle length calculator and a machine learning (ML) model 124. The ML model 124 can be trained to determine one or more categories of signal features from the heartbeat intervals based on the signal parameters 108 that have been determined. The categories of signal features can include normal cardiac rhythm and one or more arrhythmia conditions.

For example, the ML model 124 can include any of an artificial neural network (ANN) algorithm, a support-vector machine (SVM) algorithm, a decision tree algorithm, a recurrent neural network (RNN) algorithm, and a convolutional neural network (CNN) algorithm. Other types of machine learning can be used in other examples. The ML model 124 can be trained on prior EP signals measured invasively and/or non-invasively from a patient population representative of one or more known categories of signal features during a respective time interval or over a series of time intervals. Additionally or alternatively, the ML model 124 can be trained on prior EP signal reconstructed (e.g., by respective instances of reconstruction engine 46) on to a respective surface of interest from a patient population representative of one or more known categories of signal features. In a further example, one or more ML models 124 can be configured to process resulting graphical maps, consistent with how the ML model 124 is trained to identify respective trained categories of arrhythmias or a normal rhythm. As a further example, the ML model 124 can be configured to evaluate respective input maps and label respective maps that are highly correlated (e.g., similar) to known training data to classify respective signals across a surface of interest into one or more categories, which can include a normal cardiac rhythm or one or more types of arrhythmias. The input maps can be a set of one or more maps generated by output generator 42 based on the EP data 28 and geometry data 38 for a patient over one or more time intervals.

The feature calculator 66 can store the determined signal features in memory. A feature analyzer 126 can be programmed to analyze the determined features over time to determine an indication of changes over time. For example, the feature analyzer 126 can determine a change between successive heartbeat intervals or over a larger period of time that includes a plurality of heartbeat intervals. As another example, the feature analyzer 126 can determine a rate of change for a respective feature over time such as by analyzing a plurality of heartbeat intervals and a respective feature thereof. The feature analyzer 126 can provide the determined indication of changes in the respective features to the control function 40. As described herein, the control function 40 can trigger signal processing function 44 and/or the output generator 42 in response to the analysis of the determined features provided by the feature calculator 66. The control function 40 could also trigger an update to the geometry data 38 based on anatomical changes such as due to a change in intracardiac pressure, volume changes or other structural changes that could occur during a procedure. The changes in geometry data 38 further could trigger output generator 42 to regenerate a map to reflect the structural changes that are detected.

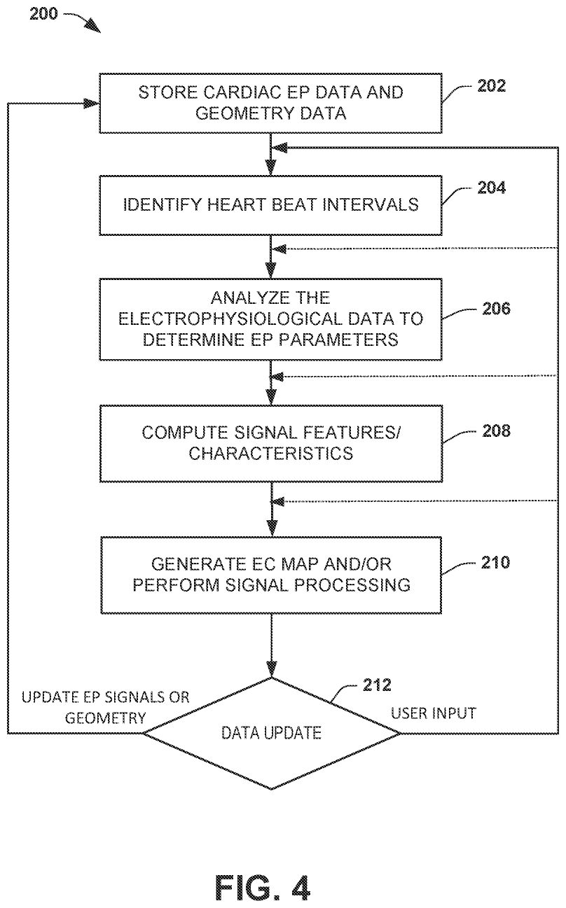

FIG. 4 depicts an example of a method 200 such that can be implemented by the system 10 to perform the functions herein. While for purposes of simplicity of explanation, the example method of FIG. 4 is shown and described as executing serially, the example method 200 is not limited by the illustrated order, as some actions could in other examples occur in different orders, multiple times and/or concurrently from that shown and described herein. Additionally, the method 200 can be implemented as machine-readable instructions executed by a processor, such as by the mapping system 30. Accordingly, the description of FIG. 4 also refers to FIGS. 1-3.

The method begins at 202 in which cardiac EP data and geometry data are stored (e.g., EP data 28 and geometry data 38 are acquired and stored in memory). At 204, respective heartbeat intervals are identified (e.g., by beat detector 62), such as based on EP data representative of cardiac EP signals measured over a time interval. The identified beats can be used to tag some or all of the EP data 28 to specify one or beats for further analysis.

At 206, the method includes analyzing a portion of the EP data (e.g., as defined by a plurality of the respective heartbeat intervals) to determine one or more parameters associated with the cardiac EP signals over the time interval. In an example, the heartbeat intervals can be detected automatically and stored as beat data with the EP data to specify the detected heartbeat intervals for the respective cardiac electrophysiological signals. The EP signals on the surface of interest, as represented in the EP data, can include respective EP signals measured invasively from the surface of interest and/or measured non-invasively from an outer body surface. In another example, additionally or alternatively, EP signals on the surface of interest, as represented in the EP data, includes reconstructed electrophysiological signals, which are calculated for the surface of interest by solving the inverse problem based on non-invasively measured electrophysiological signals and/or invasively measured signals.

At 208, the method includes computing signal features associated with the cardiac electrophysiological signals over at least a portion of the time interval based on the one or more parameters. In an example, the signal features are computed (e.g., by feature calculator 66) based on changes in respective signal features over at least a portion of the time interval based on analyzing the one or more parameters for at least two samples over time interval. For example, a graphical map can be generated (e.g., by output generator 42) responsive to the computed changes in the signal features indicating an instability and/or an arrhythmogenic condition. Alternatively, automated signal processing can be performed (e.g., by signal processing function 26 and 44) responsive to the computed changes in the signal features indicating a stable rhythm and/or a non-arrhythmogenic condition. In some examples, the signal features associated with the cardiac electrophysiological signals can be computed by applying a trained ML model to the one or more parameters determined for the portion of the electrophysiological data to ascertain the respective signal features associated with the cardiac EP signals. As described herein, the signal can include a cardiac rhythm and/or cycle length for electrophysiological signals distributed across a surface of interest.

At 210, the method includes generating a map on a surface of interest and/or performing automated signal processing based on the cardiac electrophysiological signals for heartbeat intervals that include the computed signal features (at 208). As described herein, the map can be generated automatically responsive to computing the signal features and/or the other automated signal processing can be performed automatically responsive to computing the signal features. The automated signal processing at 210 can include one or more of identifying one or more bad measurement channels, applying signal filtering or performing inverse reconstruction of electrophysiological signals on the surface of interest based on the electrophysiological data.

At 212, the method determines whether any data has been updated. If the EP signals (e.g., measured and/or reconstructed) have been updated, the method returns to 202 to repeat the method. In response to receiving a user input, in which instructions or commands are updated to control one or more aspects of the method 200, the method returns to preceding part of the method to repeat the remaining portions. The location to where the method returns can vary depending on the user input instruction that is received (as shown by dotted lines in FIG. 4).

In some examples, the method 200 can also include generating a report summarizing steps performed in the method. The report thus can include data describing each of the computed signal features and/or changes thereof associated with the cardiac electrophysiological signals over at least a portion of the time interval. The report data can be stored in memory.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A computer-implemented method comprising:
identifying respective heartbeat intervals based on electrophysiological data representative of cardiac electrophysiological signals measured over a time interval;
analyzing the cardiac electrophysiological signals over at least a portion of the time interval; and
generating a map on a surface of interest and/or performing automated signal processing based on the cardiac electrophysiological signals for heartbeat intervals, wherein the map is generated and/or the automated signal processing is performed automatically responsive to the analysis of the cardiac electrophysiological signals.

2. The method of claim 1, wherein analyzing the cardiac electrophysiological signals comprises computing changes in signal features and/or signal parameters over the at least a portion of the time interval.

3. The method of claim 2, wherein computing the changes comprises determining a difference between the signal features and/or signal parameters and a respective baseline value.

4. The method of claim 2, wherein computing the changes comprises comparing the signal features and/or signal parameters relative to a threshold.

5. The method of claim 2, wherein the map is generated responsive to the computed changes in the signal features indicating an instability and/or an arrhythmogenic condition.

6. The method of claim 2, wherein the automated signal processing is performed responsive to the computed changes in the signal features indicating a stable rhythm and/or a non-arrhythmogenic condition.

7. The method of claim 2, comprising generating the map on the surface of interest automatically responsive to the computed changes in signal features and/or signal parameters changes; and
comparing the generated map with respect to a reference map and identifying regions of interest and/or differences between the automatically generated map and the reference map, the reference map being generated for the surface of interest based on cardiac electrophysiological signals for one or more intervals that do not include the portion of the time interval for which the changes were detected.

8. The method of claim 2, further comprising generating a notification to instruct the user to move an invasive device within the patient's body responsive to the computed changes in signal features and/or signal parameters.

9. The method of claim 1, wherein computing the signal features associated with the cardiac electrophysiological signals comprises applying a trained machine learning model to the one or more parameters determined for the portion of the electrophysiological data to ascertain the signal features associated with the cardiac electrophysiological signals for the portion of the time interval.

10. The method of claim 1, wherein the signal features associated with the cardiac electrophysiological signals include a cardiac rhythm for electrophysiological signals at locations on the surface of interest.

11. The method of claim 1, wherein the signal features associated with the cardiac electrophysiological signals include at least one of cycle length, morphology, and frequency content for electrophysiological signals distributed across a surface of interest.

12. The method of of claim 1, wherein performing other automated signal processing comprises at least one of identifying one or more bad measurement channels, applying signal filtering or performing inverse reconstruction of electrophysiological signals on the surface of interest based on the electrophysiological data.

13. The method of of claim 1, wherein the electrophysiological signals on the surface of interest comprise reconstructed electrophysiological signals calculated for the surface of interest by solving an inverse problem based on the electrophysiological data and geometry data.

14. The method of claim 1, wherein the electrophysiological signals on the surface of interest comprise respective electrophysiological signals measured invasively from the surface of interest.

15. The method of claim 1, further comprising automatically detecting the heartbeat intervals in the cardiac electrophysiological signals, and storing beat data with the electrophysiological data to specify the detected heartbeat intervals for the respective cardiac electrophysiological signals.

16. The method of claim 1, further comprising:
generating a report summarizing steps performed in the method, including data describing respective computed signal features and/or changes thereof associated with the cardiac electrophysiological signals over at least a portion of the time interval; and
storing the report in memory.

17. One or more non-transitory machine-readable medium configured to store instructions, which are executable by a processor to perform the method of claim 1.

18. A system comprising:
a sensing system including one or more electrodes adapted to measure cardiac electrophysiological signals;
a computing apparatus including non-transitory memory to store data and instructions executable by a processor thereof, the data including:
electrophysiological data representative of the measured cardiac electrophysiological signals measured over at least one time interval;
geometry data representing anatomy of a patient spatially, and locations of the electrodes in three-dimensional space; and
the instructions programmed to perform a method comprising:
analyzing the electrophysiological data defined by a plurality of the respective heartbeat intervals over at least a portion of the time interval; and
generating a map on a surface of interest and/or performing automated signal processing based on the cardiac electrophysiological signals for heartbeat intervals, wherein the map is generated and/or the automated signal processing is performed automatically responsive to the analysis of the electrophysiological data.

19. The system of claim 18, wherein the sensing system includes an arrangement of body surface electrodes adapted to measure electrophysiological signals on an outer surface of a patient's body.

20. The system of claim 19, wherein the sensing system includes an invasive electrode adapted to measure the electrophysiological signals within the patient's body.

21. The system of claim 19, wherein the instructions further comprise code to reconstruct electrophysiological signals on nodes distributed across a surface of interest within the patient's body based on the electrophysiological data and the geometry data, the electrophysiological data representing the electrophysiological signals measured at the outer surface of a patient's body and within the patient's body.

22. The system of claim 18, wherein computing signal features comprises computing changes in signal features and/or signal parameters determined based on the analysis of the electrophysiological data over multiple time intervals.

23. The system of claim 22, wherein the changes are computed based on a difference between the signal features and/or signal parameters and a respective baseline value thereof.

24. The method of claim 22, wherein the changes are computed based on comparing the signal features and/or signal parameters relative to a threshold.

25. The system of claim 22, wherein the map is generated responsive to the computed changes in the signal features indicating an instability and/or an arrhythmogenic condition.

26. The system of claim 22, wherein the automated signal processing is performed responsive to the computed changes in the signal features indicating a stable rhythm and/or a non-arrhythmogenic condition.

27. The system of claim 22, further comprising instructions programmed to apply a trained machine learning model to one or more signal parameters determined for the portion of the electrophysiological data to ascertain the signal features associated with the cardiac electrophysiological signals for the portion of the time interval.

28. The system of claim 27, wherein the signal features associated with the cardiac electrophysiological signals include a cardiac rhythm for electrophysiological signals distributed across a surface of interest.

29. The system of claim 22, wherein the signal features associated with the cardiac electrophysiological signals include at least one of cycle length, morphology, and frequency content for electrophysiological signals distributed across a surface of interest.

30. The system of claim 22, wherein performing other automated signal processing comprises at least one of identifying one or more bad measurement channels in the sensing system, applying signal filtering, or performing inverse reconstruction of electrophysiological signals on the surface of interest based on the electrophysiological data.

31. The system of claim 22, wherein the instructions are further programmed to automatically detect the heartbeat intervals in the cardiac electrophysiological signals, and storing beat data with the electrophysiological data to specify the detected heartbeat intervals for the respective cardiac electrophysiological signals.

32. The system of claim 22, wherein the instructions are further programmed to:
generate a report summarizing steps performed in the method, including data describing respective computed signal features and/or changes thereof associated with the cardiac electrophysiological signals over at least a portion of the time interval; and
store the report in memory.

33. The system of claim 22, wherein the instructions are further programmed to:
generate the map on the surface of interest automatically responsive to the computed changes in signal features and/or signal parameters changes; and
compare the generated map with respect to a reference map and identify regions of interest and/or differences between the automatically generated map and the reference map based on the comparison, the reference map being generated for the surface of interest based on cardiac electrophysiological signals for one or more intervals that do not include the portion of the time interval for which the changes were detected.

34. The system of claim 22, further comprising generating a notification to instruct the user to move an invasive device within the patient's body responsive to the computed changes in signal features and/or signal parameters.
